(19) United States
(12) Patent Application Publication
(10) Pub. No.: US 2005/0053523 A1
(43) Pub. Date: Mar. 10, 2005

(54) Cell phone alcohol detector

(75) Inventor: Richard B. Brooke, Coeur d'Alene, ID (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Oxyfresh Worldwide, Inc., Coeur d'Alene, ID (US)

(21) Appl. No.: 10/769,000

(22) Filed: Jan. 30, 2004

Related U.S. Application Data
(63) Continuation-in-part of application No. 10/659,099, filed on Sep. 10, 2003.

(51) Int. Cl. G01N 33/00

(52) U.S. Cl. 422/68.1

(57) ABSTRACT

The cellular telephone/breath analyzer combination increases the functionality of the cellular telephone by informing the user of his/her breath alcohol concentration. The visual display of the cellular telephone displays the breath analysis function. Using the keypad of the cellular telephone, the user can instruct the cellular telephone/breath analyzer to perform a breath analysis. When the user exhales into the breath analyzer, a microcontroller and digital processor disposed within the cellular telephone convert the ethanol gas sensor reading into a breath alcohol concentration indicator on the visual display of the cellular telephone.

### CROSS-REFERENCE TO RELATED APPLICATION(S)

This application is related to and a continuation-in-part of the application Ser. No. 10/659,099, filed on Sep. 10, 2003, and entitled “Cell Phone/Breath Analyzer”, which is herein incorporated by reference.

### BACKGROUND OF THE INVENTION

The present invention relates to a breath sensor disposed within a portable communications device. In particular, the present invention relates to a breath analyzer for use with a cellular telephone.

The use of cellular telephones is widespread, seemingly with no end to their utility and proliferation in sight. At the touch of a keypad, verbal information can be communicated to parties located at great distances from one another and global information can be accessed from the Internet from nearly any location. Features such as caller ID, voice messaging, appointment books and calculators have added to the convenience and utility of cellular telephones.

The purpose of breath alcohol detectors is to provide the user with an indication of alcohol concentration in expired breath. The concentration of alcohol in a person's expired breath is directly correlated to the person's blood alcohol level. Due to the correlation between breath alcohol concentration and blood alcohol level, breath alcohol concentration can be used as a relatively unobtrusive aid in estimating expected functional impairment resulting from alcohol consumption. A user's breath alcohol concentration is detected using an ethanol gas sensor containing an integrated heating element.

Thus, there is a need in the art for cellular telephones that can analyze the user's breath alcohol concentration.

### BRIEF SUMMARY OF THE INVENTION

A combination cellular telephone and breath analyzer of the present invention provides the added functionality of breath analysis to the cellular telephone. The visual display of the cellular telephone provides the user with the option of having the quality of his/her breath analyzed. The user selects this function by pressing the keypad of the cellular telephone and exhaling into a breath analyzer incorporated into the cellular telephone. Once activated, the breath analyzer senses the presence of ethanol gas with an ethanol gas sensor and utilizes the measured gas concentration as an indicator of breath alcohol concentration. The breath alcohol concentration is then conveyed to the user on the visual display of the cellular telephone.

### BRIEF DESCRIPTION OF THE DRAWINGS

FIG. 1 is a front-perspective view showing the cellular telephone and breath analyzer combination of the present invention.

FIG. 2 is a block diagram showing a first embodiment of the electronics of the cellular telephone and breath analyzer combination of the present invention.

FIG. 3 is a block diagram showing a second embodiment of the electronics of the cellular telephone and breath analyzer combination of the present invention.

While the above-identified figures set forth preferred embodiments of the invention, other embodiments are also contemplated, as noted in the discussion. In all cases, this disclosure presents the present invention by way of representation and not limitation. It should be understood that numerous other modifications and embodiments can be devised by those skilled in the art which fall within the scope and spirit of the principles of this invention.

### DETAILED DESCRIPTION

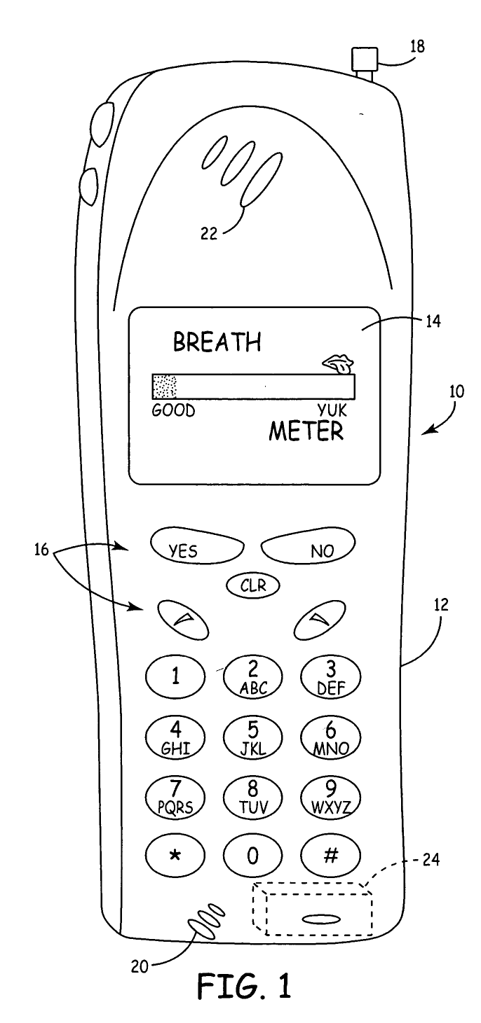

FIG. 1 depicts a typical cellular telephone 10 used to transmit and receive communication signals. The cellular telephone 10 includes a housing 12 sized to fit a human hand that houses conventional electronics for a cellular telephone including a visual display 14, keypad 16, antenna 18, battery (not shown), microphone 20 to receive sound signals, and speaker 22 to provide audio signals to the user. It is common in the art for the visual display 14 to be either a liquid crystal display (LCD) or a plasma display. The display 14 visually informs the user of the various functions available on the cellular telephone 10. The user then provides input to the cellular telephone 10 to perform a particular function by utilizing the keypad 16 to select the desired function from the visual display 14. The housing 12 of the cellular telephone 10 also includes a breath analyzer 24 within it.

The breath analyzer 24 is utilized for detecting the presence of volatile components in a user's breath. For convenience, the breath analyzer 24 is disposed in the general area of the microphone 20. The breath analyzer 24 can be linked to a distinct and separate circuit for converting the analog signal to a digital signal or to the same circuit board as the cellular telephone 10 with the cellular telephone 10 circuit board accepting signals from the breath analyzer 24. The circuit board of the cellular telephone 10 includes analog-to-digital and digital-to-analog conversion chips that translate outgoing audio signals from analog to digital and incoming signals from digital back to analog. Such chips in the cellular telephone 10 could also be used to convert the signal from the breath sensor 24.

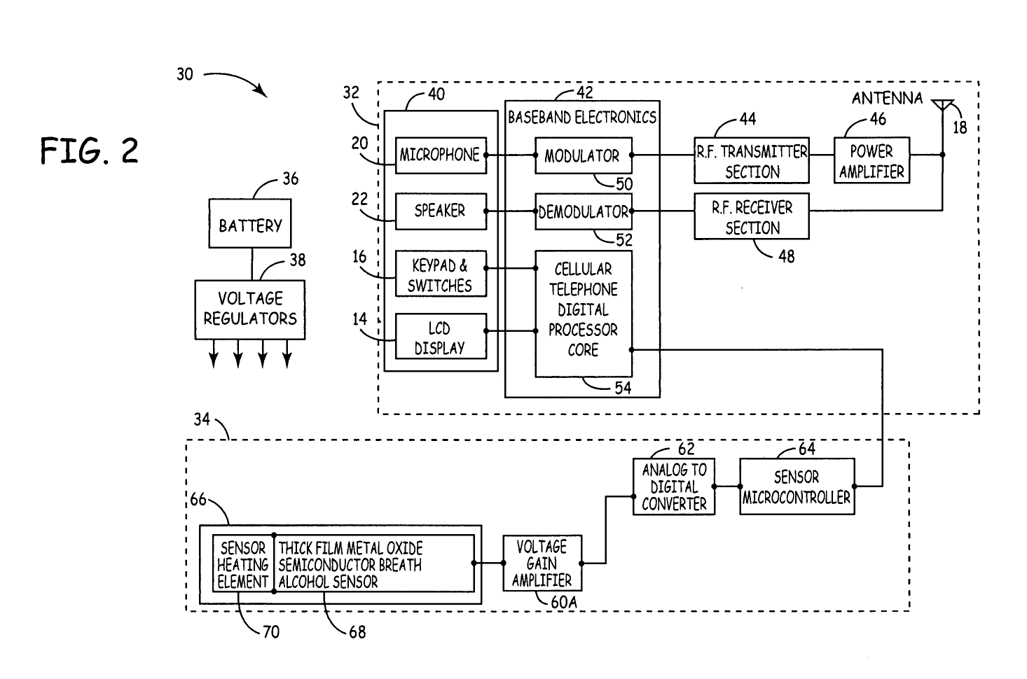

FIG. 2 depicts a block diagram of a first embodiment of the circuitry of a cellular telephone/breath analyzer combination 30. As indicated in the block diagram, the implementation of the cellular telephone/breath analyzer 30 is partitioned into two sections: a cellular telephone section 32 and a breath analyzer section 34. In addition, a battery 36 and one or more voltage regulators 38 generate power supply voltages for operation of the cellular telephone/breath analyzer 30 electronics. In order to maximize battery life, both the cellular telephone section 32 and the breath analyzer section 34 of the electronics can be switched to minimum power consumption modes when not in use.

The cellular telephone section 32 of the invention comprises a user interface 40, baseband electronics module 42, radio frequency (R.F.) transmitter 44, power amplifier 46, radio frequency receiver 48, and antenna 18. The user interface 40 includes the microphone 20, speaker 22, keypad 16, and display 14. The baseband electronics module 42 includes modulator 50 and demodulator 52 electronics and a cellular telephone digital processor core 54.

The cellular telephone digital processor core 54 is operatively connected to the keypad and switch 16, which may be used to provide input to the cellular telephone section 32. The digital processor core 54 is also operatively connected to the display 14, and may optionally be operatively connected to the microphone 20 and to the speaker 22. In this way, the digital processor core 54 is configured to present visual information on the display 14 as well as provide audio indicators through the speaker 22. The microphone 20 may also be connected to the digital processor core 54 to allow voice activation of various features of the cellular telephone 10.

The digital processor 54 also controls the sequence of events when the user communicates using the cellular telephone 10. For instance, the digital processor 54 includes modulator 50 and demodulator 52, and controls the sequence of events when the user verbally communicates into the cellular telephone 10 through the microphone 20. Audio inputs from the microphone 20 are translated into a format suitable for transmission by the modulator 50, converted to a radio frequency signal in the radio frequency transmitter section 44, power boosted by the power amplifier 46, and transmitted through the antenna 18. The radio frequency receiver section 48 amplifies incoming radio signals and converts them into a format that the demodulator 52 can use to generate analog voltage level signals that drive the speaker 22 with audio tone and reconstructed voice information.

In the first embodiment, the breath analyzer section 34 of the invention comprises an ethanol gas sensor 66 that is sensitive to ethanol gas, voltage gain amplifier 60A, analog-to-digital converter 62, and breath analyzer microcontroller 64. The breath analyzer section 34 is controlled by the breath analyzer microcontroller 64. The ethanol gas sensor 66 comprises a thick film metal oxide semiconductor 68 and an integrated heating element 70. Due to the small physical size of the breath analyzer 24, the heating element 70 requires relatively low power. When a measurement of breath alcohol concentration is desired, the integrated heating element 70 is turned on for a short period of time in order to heat the thick film metal oxide semiconductor 68. When ethanol gas is detected by the thick film metal oxide semiconductor 68 in a user's expired breath, the conductivity of the thick film metal oxide semiconductor 68 increases at a level relative to the concentration of ethanol gas in the air. An increase in the conductivity of the thick film metal oxide semiconductor 68 is detected as a voltage change that is boosted by the voltage gain amplifier 60A to a level that can be converted to digital form by the analog-to-digital converter 62. The output of the analog-to-digital converter 62 is transferred to the breath analyzer mictrocontroller 64 for scaling as an indicator of breath alcohol concentration. A measurement of low or no ethanol gas concentration is indicated as low breath alcohol concentration. A measurement of high ethanol gas concentration is indicated as high breath alcohol concentration. Measurements between low and high ethanol gas concentrations may also be indicated on a relative breath alcohol concentration scale between the two extremes.

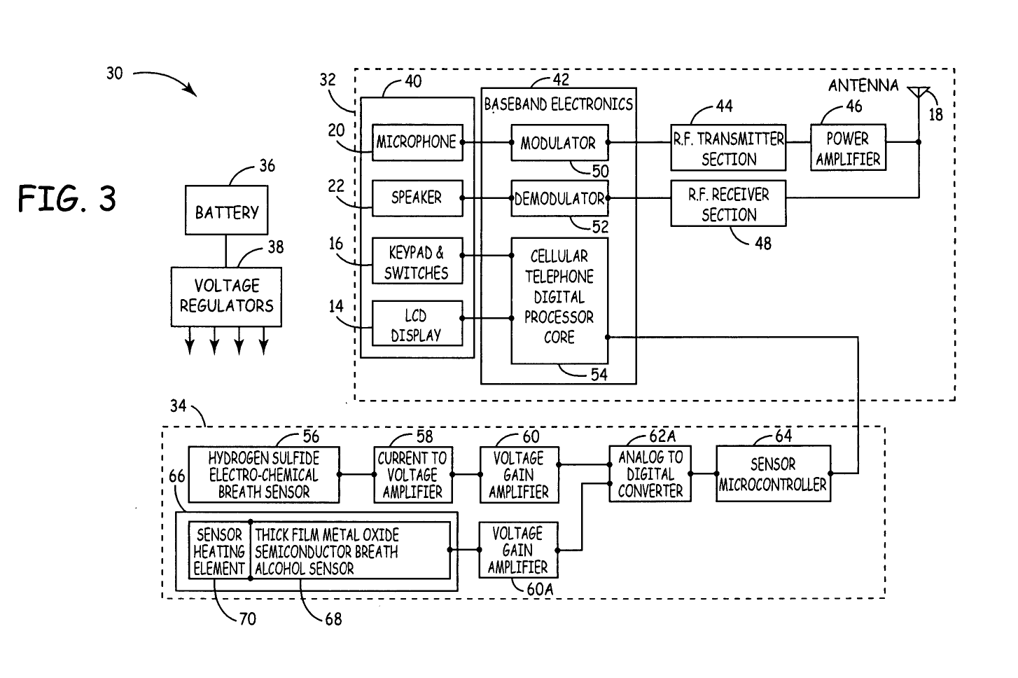

FIG. 3 depicts a block diagram of a second embodiment of the circuitry of the cellular telephone/breath analyzer combination 30. As indicated in the block diagram, the cellular telephone/breath analyzer 30 is partitioned into two sections: a cellular telephone section 32 and a breath analyzer section 34. The battery 36, voltage regulators 38, and cellular telephone section 32 of the second embodiment maintains the same configuration as the cellular telephone section 32 of the first embodiment.

In the second embodiment, the breath analyzer section 34 can perform both a breath quality analysis and a breath alcohol concentration analysis. The breath analyzer section 34 comprises a breath analyzer microcontroller 64, multiplexed input analog-to-digital converter 62A, and electrochemical gas sensor 56 and ethanol gas sensor 66 with their associated signal processing electronics. The breath quality analysis and breath alcohol concentration analysis functions are controlled by the breath analyzer microcontroller 64. It is a common practice for a single microcontroller to control multiple functions such as breath quality analysis and breath alcohol concentration analysis. It is also common to use a single analog-to-digital converter with multiplexed inputs from a number of analog input sources.

The electronics necessary to perform a breath quality analysis comprise an electrochemical sensor 56 that is sensitive to hydrogen sulfide gas, current-to-voltage amplifier 58, and voltage gain amplifier 60 coupled to one of the multiplexed inputs of the analog-to-digital converter 62A. When hydrogen sulfide gas is detected by the electrochemical sensor 56, the electrochemical sensor 56 produces a current proportional to the concentration of hydrogen sulfide gas in the current-to-voltage amplifier 58. The output of the current-to-voltage amplifier 58 is then boosted by the voltage gain amplifier 60 to a level that allows the analog-to-digital converter 62A to convert this signal from analog to digital form. The output of the analog-to-digital converter 62A is transferred to the breath analyzer microcontroller 64 for scaling as an indicator of breath quality. A measurement of low or no hydrogen sulfide gas concentration is indicated as poor breath quality. A measurement of high hydrogen sulfide gas concentration is indicated as poor breath quality. Measurements between low and high hydrogen sulfide concentrations may also be indicated on a relative breath quality scale between the two extremes.

The electronics necessary to perform a breath alcohol concentration analysis comprise an ethanol gas sensor 66 and a voltage gain amplifier 60A coupled to one of the multiplexed inputs of the analog-to-digital converter 62A. The ethanol gas sensor 66 comprises a thick film metal oxide semiconductor 68 and an integrated heating element 70. Due to the small physical size of the breath analyzer 24, the heating element 70 requires relatively low power. When a measurement of breath alcohol concentration is desired, the integrated heating element 70 is turned on for a short period of time in order to heat the thick film metal oxide semiconductor 68. When ethanol gas is detected by the thick film metal oxide semiconductor 68 in a user's expired breath, the conductivity of the thick film metal oxide semiconductor 68 increases at a level relative to the concentration of ethanol gas in the air. An increase in the conductivity of the thick film metal oxide semiconductor 68 is detected as a voltage change that is boosted by the voltage gain amplifier 60A to a level that can be converted from analog to digital form by the analog-to-digital converter 62A. The output of the analog-to-digital converter 62A is transferred to the breath analyzer microcontroller 64 for scaling as an indicator of breath alcohol concentration. A measurement of low or no ethanol gas concentration is indicated as low breath alcohol concentration. A measurement of high ethanol gas concentration is indicated as high breath alcohol concentration. Measurements between low and high ethanol gas concentrations may also be indicated on a relative breath alcohol concentration scale between the two extremes.

The expected useful lifetime of an ethanol gas sensor or an electrochemical sensor is two to three years. In one embodiment, the ethanol gas sensor 68 element or electrochemical sensor 56 element can be constructed as a user replaceable module. The breath analyzer microcontroller 64 can be programmed to monitor the condition of the ethanol gas sensor 68 or electrochemical sensor 56 and alert the user when it needs to be replaced.

The breath analyzer section 34 is operatively connected to and is also controlled by the cellular telephone digital processor core 54. For instance, the interface between the cellular telephone section 32 and the breath analyzer section 34 may be a bi-directional digital serial data communications link between the cellular telephone digital processor core 54 and the breath analyzer microcontroller 64. In this embodiment, the serial data communications link utilizes a UART (universal asynchronous receiver transmitter) for full duplex serial data transfer. The UART is a logical choice for this design because many microcontrollers and other digital processors are available with these devices built in. However, those skilled in the art will recognize that there are many types of serial data communication links that can be used.

In operation, the user selects the breath analyzer function using the keypad 16. The cellular telephone digital processor 54 then issues a request for a breath analysis to the breath analyzer microcontroller 64, such as by serial communications interface, and instructs the user either audibly via the speaker 22 or visually via the display 14 to exhale into the breath analyzer 24 on the face of the cellular telephone 10. Once the breath analysis is completed, the breath analyzer microcontroller 64 transmits the results of the analysis by the serial communications interface to the cellular telephone digital processor 54. The digital processor 54 then outputs the analysis results, such as via the visual display 14 or via the speaker 22.

The design of the cellular telephone section 32 in both embodiments presented is not intended to be an exhaustive description of cellular telephone technology. The simplified description of electronics circuitry representative of a cellular telephone is presented as an aid to understanding the invention. This design embodiment maintains a clear separation between the cellular telephone section 32 and breath analyzer section 34 in order to clearly describe the unique features of the invention. As an alternative embodiment, the cellular telephone section 32 and breath analyzer section 34 may be integrated into a single unit to minimize physical size and to reduce manufacturing cost. In addition, the breath analyzer section 34 analog-to-digital converter 62 and breath analyzer microcontroller 64 functions could be integrated into the cellular telephone digital processor core 54. Under this embodiment, the interface between the breath analyzer and cellular telephone sections 32, 34 would be the analog voltage output of the breath analyzer section 34 voltage gain amplifier 60.

Although the present invention has been described with reference to preferred embodiments, workers skilled in the art will recognize that changes may be made in form and detail without departing from the spirit and scope of the invention.

1. A portable communications device for sending and receiving verbal communications having a housing sized to fit a human hand, characterized by:
a breath analyzer within the housing for sensing volatile components in a user's breath and providing a breath analyzer output based upon the sensed volatile components; and
a user interface carried by the housing for providing an output based upon results of a breath test performed by the breath analyzer.

2. The portable communications device of claim 1, wherein the volatile components in the user's breath sensed by the breath analyzer is ethanol gas.

3. The portable communications device of claim 2, the breath analyzer comprising an ethanol gas sensor for sensing ethanol gas in the user's breath and providing an ethanol gas sensor output based upon the sensed ethanol gas.

4. The portable communications device of claim 3, wherein the ethanol gas sensor comprises:
a thick film metal oxide semiconductor; and
a heating element for heating the thick film metal oxide semiconductor.

5. The portable communications device of claim 4, wherein the breath analyzer further comprises a voltage gain amplifier.

6. The portable communications device of claim 5, wherein the breath analyzer further comprises an analog-to-digital converter.

7. The portable communications device of claim 3, wherein the breath analyzer further comprises a controller for scaling the ethanol gas sensor output.

8. The portable communications device of claim 1 and further comprising input for activating the breath analyzer.

9. The portable communications device of claim 1, wherein the user interface is a visual display.

10. The portable communications device of claim 9, wherein the visual display is a liquid crystal display.

11. The portable communications device of claim 9 wherein the visual display is a plasma display.

12. The portable communications device of claim 1, wherein the user interface is a speaker.

13. A cellular telephone, an improvement comprising:
a user input to select a breath analysis;
a breath analyzer for sensing volatile components in a user's breath and providing a breath analyzer output based upon the sensed volatile components; and
a processor for processing the breath analyzer output and providing a user perceivable output reporting the breath analysis based on the processed breath analyzer output.

14. The cellular telephone of claim 13, wherein the volatile components sensed in the user's breath sensed by the breath analyzer is ethanol gas.

15. The cellular telephone of claim 14, wherein the breath analyzer further comprises an ethanol gas sensor for sensing ethanol gas in a user's breath.

16. The cellular telephone of claim 15, wherein the ethanol gas sensor comprises:
a thick film metal oxide semiconductor; and
a heating element for heating the thick film metal oxide semiconductor.

17. The cellular telephone of claim 16, wherein the ethanol gas sensor provides an ethanol gas sensor output based upon the sensed ethanol gas.

18. The cellular telephone of claim 17, wherein the breath analyzer further comprises a controller for scaling the ethanol gas sensor output.

19. The cellular telephone of claim 18, wherein the breath analyzer further comprises a voltage gain amplifier.

20. The cellular telephone of claim 19, wherein the breath analyzer further comprises an analog-to-digital converter.

21. The cellular telephone of claim 13, wherein the user perceivable output is a visual display.

22. The cellular telephone of claim 21, wherein the visual display is a liquid crystal display.

23. The cellular telephone of claim 22, wherein the visual display is a plasma display.

24. The cellular telephone of claim 13, wherein the user perceivable output is an audible indicator.

25. A portable communications device comprising:
a keypad for selecting a breath analysis;
an antenna;
a microphone for receiving verbal signals from a user;
a speaker for providing audio signals to the user;
communications electronics connected to the microphone, the speaker and the antenna for transmitting and receiving communications signals;
a visual display for providing a visual output;
a breath analyzer for sensing volatile components in a user's breath and providing a breath analyzer output based upon the sensed volatile components; and
a digital processor for controlling the visual display to provide a visual output based on the breath analyzer output.

26. The portable communications device of claim 25, wherein the volatile components in the user's breath sensed by the breath analyzer is ethanol gas.

27. The portable communications device of claim 26, the breath analyzer comprising an ethanol gas sensor for sensing ethanol gas and providing an ethanol gas sensor output based upon the sensed ethanol gas.

28. The portable communications device of claim 27, wherein the ethanol gas sensor comprises:
a thick film metal oxide semiconductor; and
a heating element for heating the thick film metal oxide semiconductor.

29. The portable communications device of claim 28, wherein the breath analyzer further comprises a voltage gain amplifier.

30. The portable communications device of claim 29, wherein the breath analyzer further comprises an analog-to-digital converter.

31. The portable communications device of claim 30, wherein the breath analyzer further comprises a controller for scaling the results of the ethanol gas sensor output.

32. The portable communications device of claim 25, wherein the visual display is a liquid crystal display.

33. The portable communications device of claim 25, wherein the visual display is a plasma display.

34. A portable communications device for sending and receiving verbal communications having a housing sized to fit a human hand, characterized by:
a breath analyzer within the housing for sensing volatile components in a user's breath and providing a breath analyzer output based upon the sensed volatile components; and
a user interface carried by the housing for providing an output based upon results of a breath test performed by the breath analyzer.

35. The portable communications device of claim 34, wherein one of the volatile components in the user's breath sensed by the breath analyzer is hydrogen sulfide.

36. The portable communications device of claim 35, wherein one of the volatile components in the user's breath sensed by the breath analyzer is ethanol gas.

37. The portable communications device of claim 36, the breath analyzer comprising an electrochemical sensor for sensing hydrogen sulfide in the user's breath and providing an electrochemical sensor output based upon the sensed hydrogen sulfide.

38. The portable communications device of claim 37, the breath analyzer comprising an ethanol gas sensor for sensing ethanol gas in the user's breath and providing an ethanol gas sensor output based upon the sensed ethanol gas.

39. The portable communications device of claim 38, wherein the ethanol gas sensor comprises:
a thick film metal oxide semiconductor; and
a heating element for heating the thick film metal oxide semiconductor.

40. The portable communications device of claim 39, wherein the breath analyzer further comprises a current-to-voltage amplifier and a first and a second voltage gain amplifier.

41. The portable communications device of claim 40, wherein the breath analyzer further comprises a multiplexed analog-to-digital converter.

42. The portable communications device of claim 41, wherein the breath analyzer further comprises a controller for scaling the electrochemical sensor output and the ethanol gas sensor output.

43. The portable communications device of claim 34 and further comprising input for activating the breath analyzer.

44. The portable communications device of claim 34 wherein the user interface is a visual display.

45. The portable communications device of claim 44 wherein the visual display is a liquid crystal display.

46. The portable communications device of claim 44 wherein the visual display is a plasma display.

47. The portable communications device of claim 34 wherein the user interface is a speaker.
